(19) United States
(12) Patent Application Publication
(10) Pub. No.: US 2012/0053648 A1
(43) Pub. Date: Mar. 1, 2012

(54) Systems and methods for smoking cessation.

(76) Inventors: Rolf Neher, Waldsee (DE); Heinz Schade, Reutlingen (DE); Boris Breivogel, Bad Durkheim (DE)

(21) Appl. No.: 13/291,055

(22) Filed: Nov. 7, 2011

Related U.S. Application Data
(63) Continuation-in-part of application No. 11/351,110, filed on Feb. 8, 2006.
Application No. 11/351,110 is a continuation-in-part of application No. 11/351,111, filed on Feb. 8, 2006.

(30) Foreign Application Priority Data
Feb. 8, 2005 (DE) 10 2005 007 908.3
Mar. 24, 2005 (DE) 10 2005 014 586.8

(51) Int. Cl. A61N 1/36; A61N 5/067

(52) U.S. Cl. 607/3; 607/58

(57) ABSTRACT

Addiction treatment, specifically smoking cessation, can be effectively treated utilizing stimulation of low resistance skin locations by employing a combination of an electrical current and a laser beam. In a preferred stimulation treatment, pulsed stimulation is used, stimulating skin locations in the ear and nose areas. The present stimulation treatment is simple and cost effective, lasting about 10-20 minutes per treatment session, with an average of 1 to 2 repeated treatment sessions per person. The present stimulation treatment is proven to be effective in assisting in smoking cessation, showing high percentage of long term smoking abstinence in just one treatment session.

This application is a continuation-in-part of application Ser. No. 11/351,111, filed Feb. 8, 2006, entitled “System for the stimulation of acupuncture points” and application Ser. No. 11/351,110, filed Feb. 8, 2006, entitled “System for the stimulation of acupuncture points”, which claims priority from German application No. 10 2005 014 586.8 filed Mar. 24, 2005, entitled “System zur Stimulation von Akupunkturpunkten”, which claimed priority from German application No. 10 2005 007 908.3, filed Feb. 8, 2005, entitled “System zur Stimulation von Akupunkturpunkten” which are incorporated herein by reference.

### FIELD OF THE INVENTION

This invention is related to acupuncture therapy, particularly related to the stimulation of skin locations with therapeutic properties, and particularly related to addiction treatment, specifically for smoking cessation.

### BACKGROUND OF THE INVENTION

The effectiveness of acupuncture for various illnesses has been recognized for a long time, and has been proven recently in several studies. Traditionally, specific points in the body, called acupuncture points, were reached by piercing with needles for stimulation. The stimulation can also occur from thermal energy by burning Moxa during treatment, or by manual pressure from fingertips or knuckles (acupressureo) or radiation with laser energy.

Electrical stimulation of acupuncture points is also well-known, either with alternating or direct, constant or pulsed, current or voltage applied to the piercing needles or directly to the body. In recent years, visible light and laser light are also utilized as a further enhancement of the stimulation of acupuncture points.

There are several problems with existing technologies:

The stimulation of acupuncture points with piercing needles can bring risk of infection and is frequently not acceptable, especially for children. The same applies to the stimulation of piercing needles with electrical current. Laser light stimulation with different frequencies provides a small therapeutic effect, but this effect is realized only with much experiences or practice. For electrical stimulation, there is an indeterminable variation of pulse waveforms and frequencies, and thus so far no reliable studies have admitted to the predictability of the effect of the electrical stimulation of acupuncture points. There are indirect proofs from the electrical physiology realizations over the fact that nerve actions can be blocked by high frequencies and activated by low frequencies (1-100 Hertz), however acupuncture point was not proven to be an information line over nerves. In contrary some results suggest that electric energy may be transported directly from cell to cell in line with the described meridians of traditional Chinese medicine.

Acupuncture therapy has also been used for aiding in smoking cessation, but has not shown significant results, for example, achieving six month cessation rates of between 12% and 17% after eight weeks of treatment. Recent reviews of acupuncture therapy for smoking cessation conclude that while no firm conclusions can be made due to methodological problems, there is no consistent evidence that acupuncture, acupressure, laser therapy or electrostimulation are effective. Frequent or continuous stimulation treatments are required.

Thus a simple, effective, reliable stimulation system and method for acupuncture point treatment of smoking cessation is not well known.

### SUMMARY OF THE DESCRIPTION

The present invention relates to stimulation treatments for addiction, such as assisting in smoking cessation, utilizing sequential applications of an electrical current or preferably a combined stimulation of an electrical current and a laser beam. The treatment points were developed from acupuncture experience, but the present treatment can use acupuncture points as well as areas that are not described as acupuncture points. The to-be-treated areas are typically found to have reduced skin resistance, e.g. in smokers in withdrawal or women after hysteroscopy, and can be identified with resistance measurement equipment and techniques.

In some embodiments, the present invention discloses a system and method for treating smoking addiction, either comprising identifying low resistance skin locations or using already identified skin locations, and then sequentially applying an electrical stimulation or preferably an electrical stimulation in combination with a laser light stimulation. The present invention is proven to be effective, and can achieve long term smoking cessation for an average of 1 or 2 treatment sessions.

In some embodiments, the stimulation is preferably in pulse form, such as pulsed electrical current, pulsed laser beam, or a combination of pulsed electrical current and pulsed laser beam. The electrical stimulation preferably comprises a pulse DC current having frequency between 0.5 and 2 Hz with a voltage less than 160V. The waveform of the pulse DC current preferably comprises a peak pulse between 1 and 3 mA for less than 0.2 second, preferably between 1 to 20 msec, followed by a constant current between 0.5 and 3 mA for less than 1 second, and followed by a zero current for less than 1 second. The laser light stimulation preferably comprises a pulsed red laser light having frequency less than 10 Hz and having power less than 5 mW. In some embodiments, the stimulation is performed for less than 10-20 seconds per skin location, and the stimulation session can be completed for less than 10-20 minutes.

In some embodiments, the stimulation is performed on skin locations on the ear and nose areas. The skin locations can be low resistance skin points, and Ohmic resistance measurements or acupuncture point finders can be used to find most of the treatment points. In some embodiments, the skin locations can be from a standardized set of skin points that have been found to be effective in addiction treatment. Localization and sequence of a set of found as suitable points can be displayed on a monitor with software guiding the therapist's movements from point to point in a standardized sequence. In some embodiments, the ear points are stimulated first, followed by the stimulation of nose points, for a total of 50 points or less.

The present stimulation treatment for aiding in smoking cessation is simple and inexpensive, with the number of treatment sessions varying from 1 to 5 sessions per patient. The number of treatment sessions is determined based on the patient's level of nicotine dependence, with low and medium addiction requiring less than 3 treatment sessions, and 5 treatment sessions for patients with very high addiction.

The present stimulation addiction treatment is proven to be effective for assisting in smoking cessation, especially for patients complying with the prescribed treatment schedule. A high overall success rate (˜50%) of long term smoking abstinence (˜2 years) was achieved for an average of 1 to 2 treatment sessions for patients completing only an average of one third of the prescribed treatment schedule. A higher success rate can be expected.

Other embodiments are also described.

### BRIEF DESCRIPTION OF THE DRAWINGS

FIG. 1 shows an embodiment of the present invention stimulation of acupuncture points, employing a combination of electrical current, laser beam and blue light.

FIG. 2 shows an embodiment of the electrical pulsed current.

FIG. 3 illustrates an exemplary stimulation system.

FIGS. 3A-3B illustrate exemplary stimulation systems.

FIGS. 4A-4B illustrate exemplary electrodes.

FIG. 5A illustrates an exemplary display.

FIG. 5B illustrate an exemplary stimulation processes.

FIG. 6 illustrates exemplary overview of a treatment process.

FIG. 7 illustrates an exemplary result.

### DETAILED DESCRIPTION OF THE PREFERRED EMBODIMENTS

The present invention discloses a system and method for stimulation therapy and therapeutic effect utilizing a stimulation of acupuncture and acupuncture related points. The stimulation according to the present invention comprises an electrical stimulation with or without combination with a laser beam stimulation. The stimulation is preferably in pulse form, such as pulsed electrical current or pulsed laser beam, or a combination of pulsed electrical current and pulsed laser beam.

The present invention can further comprise a stimulation point indicator to assist in the selection of acupuncture points. The stimulation point indicator preferably comprises a display showing a sequence of intended therapy-effect determined to be stimulated regions, distinguishable from other regions. The stimulation point indicator can assist the therapist, who simply uses the points and sequence given on the monitor, instead of searching or determining the stimulation points before treatment.

The present invention stimulation is preferably controlled by a computer system for reliability, repeatability, and ease of control. Plus, the present invention can be practiced by an operator with minimal training on the equipment.

In some embodiments of the invention, the system according to the present invention for the stimulation of acupuncture points comprises a power source for delivering an electric current to the acupuncture point.

In some embodiments of the invention, the system according to the present invention for the stimulation of acupuncture points preferably comprises a power source for delivering an electric current to the acupuncture point, and a laser source for emitting a laser beam, also to the same acupuncture point.

In another embodiment of the invention, the system according to the present invention for the stimulation of acupuncture points comprises a power source for delivering an electric current, a laser source for emitting a laser beam, and a combination unit to enable an overlapping delivery of a combination of the electric current and the laser beam to the acupuncture point.

In one configuration, the laser beam is a pulsed laser beam, and the laser source for emitting a laser beam is set up to emit a pulsed laser beam. The pulsed laser beam is preferably a pulsed red laser light having frequency of 12-18 cycles per second and an intensity of less than 5-10 mW. The laser beam can have different wavelengths, and the laser source for emitting a laser beam is preferably set up to emit laser beams of different wavelengths.

The power source can be electrically isolated from the main circuit, from the surrounding electrical circuits such as the control computer or the main power supply. The power source is configured for delivering an electrical current, and preferably a pulsed current. The current pulses are preferably having a frequency from 0.5 to 2 cycles per second and amplitude from 0.05 to 3 mA. The current is preferably controlled by a computer for repeatability, accuracy and ease of control.

The combination unit preferably comprises an application housing and a handle. In some embodiments, the application housing provides the mechanism for the electrical current, and preferably comprises an application electrode for delivering the current. The application electrode is connected to the power source, and the electrical current is delivered to the stimulation point through this electrode. In some embodiment, the application housing provides the mechanism for the combination of the laser beam and the electrical current, and preferably comprises an application electrode for delivering the current, with an optical beam guide in the center of the application electrode for conducting the laser light. The application electrode is connected to the power source, and the electrical current is delivered to the stimulation point through this electrode. The optical beam guide is connected to the laser source, and the laser beam is delivered to the stimulation point through this optical guide.

The present invention stimulation system can also comprise an additional source of light, arranged to illuminate the area of acupuncture points. The source of light is preferably a visible light, such as a continuous blue light. The source of light can be arranged above the application housing and radiates a continuous light, preferably blue light, to the vicinity of the acupuncture point to be stimulated. In some embodiments, the light beam is shielded from acupuncture point, illuminating only the vicinity of the acupuncture point without affecting the operation of the laser beam. The light can also serve as illumination for viewing or for a camera.

The present invention stimulation system can also comprise an additional stimulation of a DC voltage starting from 140-170 V.

In a preferred embodiment, the body of a patient is connected to the negative terminal of the power source generating the electrical current, or the negative terminal of the DC voltage.

The duration of stimulation can be calculated as a function of biometrics and personal data of the patient.

The stimulation system according to invention is preferably accomplished by a computer control for repeatability, reliability and ease of control.

The present invention can further comprise a stimulation point indicator subsystem for acupuncture point therapy in which the body regions which can be stimulated can be indicated as points, and can be distinguishable from other non-stimulationable regions. The stimulation point indicator is preferably displaying therapy adapted stimulationable acupuncture points distinguishable from other non-stimulationable points, or displaying the anatomic locations of a therapy adapted sequence of stimulationable acupuncture points distinguishable from other non-stimulationable points. A stimulation point indicator for acupuncture therapy according to the present invention can comprise indication lines identifying the location of therapy adapted acupuncture points in reference to anatomical landmarks.

The stimulation point indicator can comprise a display with computer-aided control for the display of acupuncture points. The identification of acupuncture points can be accomplished by measuring the skin impedance or resistance. It is known that acupuncture points coincide with points of low body impedance, and thus an impedance measuring system can locate points of low skin resistance or impedance for display or made audible. The stimulation point indicator according to the present invention can greatly simplify the task of the operator servicing the patient by showing on the display the areas that can be stimulated versus the areas that cannot be stimulated. The display of the stimulation point indicator can also display additionally biometrics data of the patient.

The present invention further discloses a stimulation of acupuncture points utilizing a current, or preferably a combination of a current and a laser beam and connected with a stimulation point indicator, in particular with the stimulation point indicator system as disclosed above. The acupuncture therapy by a current stimulation, or preferably a combination of a current/laser beam stimulation in connection with a stimulation point indicator is preferably controlled by a computer. The computer program according to the invention includes program codes to accomplish all the disclosed invention, if the computer program is accomplished on a computer or an appropriate arithmetic and logic unit. The present invention also covers these program codes, which are stored on a computer-readable data medium.

The present invention discloses a system as well as a method for utilizing a current, or preferably a combination of a current/laser beam stimulation. The present invention stimulation of acupuncture points by a current stimulation, or preferably a combination of electrical current stimulation and laser beam stimulation can exhibit therapeutic effect, and can be effective in treatment therapy such as stimulation therapy or acupuncture point therapy. Thus the present invention also discloses a method for stimulation therapy wherein the stimulation is a current stimulation, or preferably a combination of an electrical current stimulation and laser beam stimulation. The stimulation therapy is preferably applied to locations on the body that are usable for acupuncture, acupressure, sonopuncture and laser acupuncture treatment

A method according to the present invention for the stimulation of an acupuncture point comprises the stimulation of an acupuncture point by a current. A preferred method according to the present invention for the stimulation of an acupuncture point comprises the stimulation of an acupuncture point by a combination of a current and a laser beam.

The therapeutic method according to the present invention also preferably employs a pulsed current, a pulsed laser beam, or a combination of synchronized pulsed current and pulsed laser beam.

The therapeutic method can further comprise a step of stimulating the acupuncture point with an additional DC voltage from between 140 to 170 V. The therapeutic method can further comprise a step of calculating the duration of the stimulation based on the biometrics and personal data of the patient, and stopping the stimulation when this duration is reached. The present invention also comprises the method to display the stimulation regions as points on a display screen, and these stimulation points are distinguishable from other non-stimulationable points. The display is preferably controlled by a computer-aided system for acupuncture therapy, and can further display additional biometrics and personal data of the patient.

The invention, at least with the preferential embodiments, consists of many advantages as listed below.

The stimulations by acupuncture points according to the present invention can be accomplished by persons who do not have training in acupuncture.

The invention, in principle, can be used with all disease or addiction problems, for acupuncture therapy can be an effective treatment on many different illnesses.

The invention allows ease of production and further improvements.

The described pulse waveforms and frequencies are the optimal stimulation form and proven to be effective for preferably acupuncture points on the head according to the present invention.

In some embodiments, the present invention uses well-defined electrical current stimulations. In some embodiments, the present invention uses a combination of well-defined electrical currents together with a laser light to accomplish better stimulation sequence of acupuncture points as compared with past procedures. The guidance of the sequences as well as the control of the pulse formation and acupuncture point excitation preferably takes place via a computer program, so that a stimulation by acupuncture points is controllable and reproducible as well as optimizable.

The preferred embodiment of the present invention acupuncture therapy is controllable by screen guidance to an operator. This computer-controlled guidance also leads to the reproducibility of the treatment.

Thus an optimal electrical/laser stimulation is disclosed, which leads to provable stimulation results. Further, laser light of different colors can be used. By the electrical stimulation with or without pulsed laser light of different colors, an improvement of the stimulation results can further be accomplished.

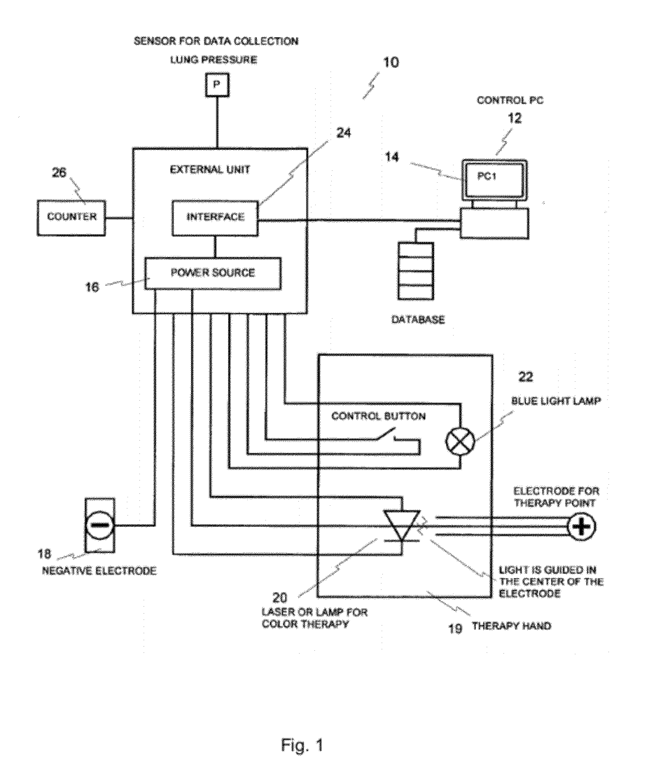

FIG. 1 represents a system 10 according to an embodiment of the present invention. It includes a computer (P/C) 12, on whose monitor 14 the points which can be stimulated are shown, and at which points the optimal stimulation can be performed. The organs illustrated on the monitor 14 are the regions to be treated or the regions where the therapeutic points are located (e.g. a knee with degenerative process in a joint, the ears, and the area of the nose wings). In addition lung function data, such as lung pressure collected from sensor P, are shown on the monitor 14. The system 10 can include a conventional impedance measuring system for locating points of low skin resistance or impedance for display on the monitor 14 or audible perception.

The control unit (computer 12 with control program and monitor 14) as shown in FIG. 1 is connected with an external unit through an interface 24. The external unit comprises a power source 16 for delivering the electrical current, and a power source with peripheral components for the laser source. The external unit is controlled by the computer 12 also through the interface 24. The power source 16 is attached to the patient by a hand electrode 18 with negative polarity. The positive terminal is implemented in the form of a combination unit 19 (therapy hand) for the user. The combination unit 19 contains a red light laser 20 (or lamp).

Description of the Combination Unit

The combination unit 19 comprises an application housing with a handle. The handle is designed for manual handling of the application housing by an operator. The application housing comprises an electrical conducting material for the electrode carrying the electrical current, and has in the center an optical conductor guide from the red laser device. The red laser light is positioned in the center of the electrode contact surface and beams direct at the patient, together with the electrical current from the electrode. The laser light is pulsed and can be switched on or off separately. Above the application housing is a source of light 22 for continuous blue light, which can also be switched on and off separately. The function of the pulsating, red laser light is an activation of the underlying acupuncture points in the skin of the patient. In certain situations, the function of the constant, blue light can be a simultaneous illumination of the surrounding acupuncture points. The electrode for therapy point possesses a spherical contact surface of approx. 5 mm in diameter. In some embodiments, the electrode can comprise a curved shape at the end, preferably larger than 1 mm, and more preferably larger than 3 mm. This ensures a safe transmission of the current pulse. The stimulation of the respective acupuncture point is started by the user by a control button at the combination unit, which is connected with the computer 12. The computer automatically controls the time of stimulation per simulation point according to the biometrics and personal data of the patient. In addition, the control button provides a means for any interruption and restart of the stimulation by the user at any time.

In some embodiments, the electrode tip is not a needle, but comprises a curved shape ball, which can relax the requirement of precise locations of the skin points to be stimulated. Thus, in some embodiments, the skin points can be located by anatomy landmark, and determined from experience. For example, the to-be-stimulated skin points can be the skin points that showed in previous trials and experiment to be useful for treating addiction, such a smoking cessation, in many individuals. The present invention found flexibility in acupuncture points, and skin points based on anatomy landmark can be used successfully.

In some embodiments, the present invention discloses a set of acupuncture points on the face, such as on the ears and nose, for stimulation treatment for many patients without the need to perform measurement or to determine the specific skin points for the individuals. In some embodiments, the flexibility in stimulation point locations can be attributed to the large tip of the stimulation electrode, relaxing the precision requirement for skin point determination.

Description of the Power Source

The stimulation system comprises a power source 16, which is connected by an interface 24 to the computer 12. The power source 16 is electrically isolated from the surrounding circuits and from the computer 12. It can have voltage and current limiters, generating a maximum voltage and a maximum current. The computer 12 controls the magnitude of the current, the duration and the waveform of the current pulse as well as the duration of the synchronous, pulsed laser beam. This control is passed on to the combination unit 19. At the computer interface, sensors (e.g. lung pressure P, pressure sensors for breath current measurements, CO measurement, and/or other measuring instrument) are also attached for the collection by biometrics data.

Description of the Stimulation Point Indicator

The user enters first the numeric data of the patient relevant to the stimulation, (e.g., age, average smoked cigarettes per day in the last month, etc.). Additionally biometrics measured values can be displayed over the interface 24 (e.g. max. lung pressure P). The software computed the optimal duration of the stimulation from the biometrics measured values and the entered patient data and then displays this information on the monitor 14. The stimulation treating stops the current intensity and/or the laser beam when reaching the optimal duration as calculated and shown on the monitor 14. The monitor screen surface shows the relevant regions (e.g. ears, nose, knee joints etc.) with the points which can be stimulated. The points that can be stimulated in each case are indicated distinctive to the other non-stimulationable points on the monitor 14. The stimulation of the succession of the points is consistently controlled by the software. A mechanical counter 26 can be used to control the number of completed stimulation sequences. Thus the number of accomplished stimulations can be ascertained.

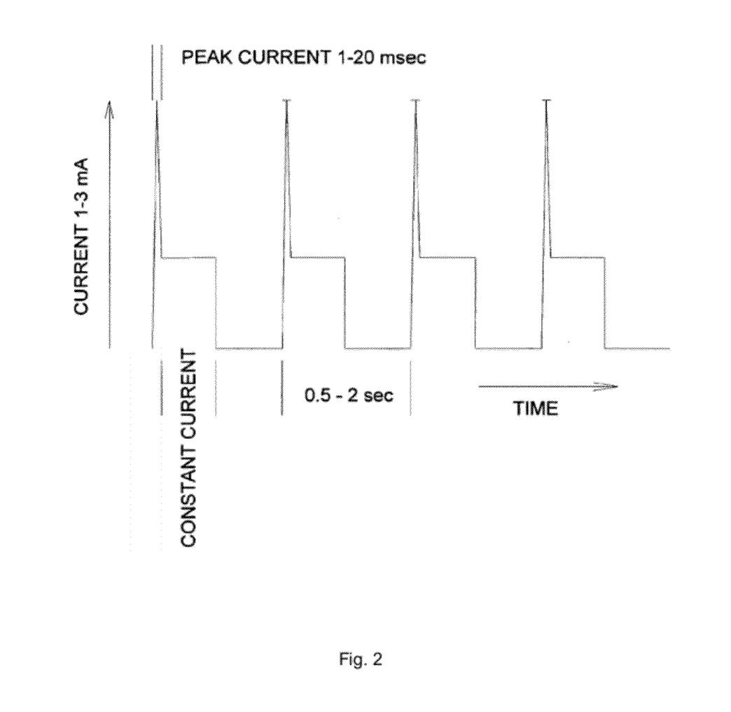

FIG. 2 shows a preferred embodiment of the electrical current waveform of one impulse. The period of the electrical current is about 0.5 to 2 sec, with a peak current of less than 20 msec, preferably between 1 to 20 msec. The peak current is preferred less than 3 mA in magnitude, preferably between 0.05 and 3 mA. A constant current is then applied after the initial peak current. The constant current can last for the rest of the period, or can last only partially (about half of the period as shown in FIG. 2), with another constant current (preferably zero current) for the rest of the period.

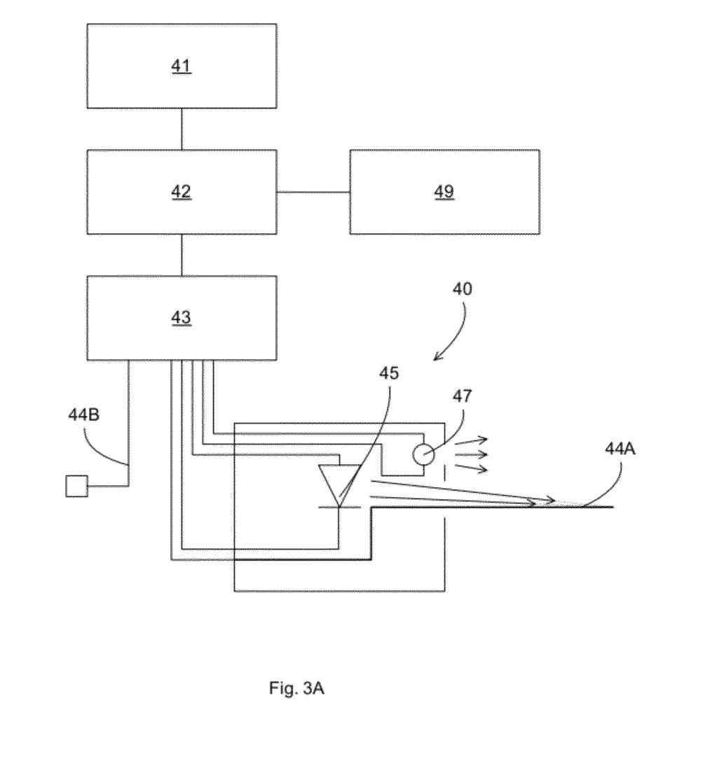

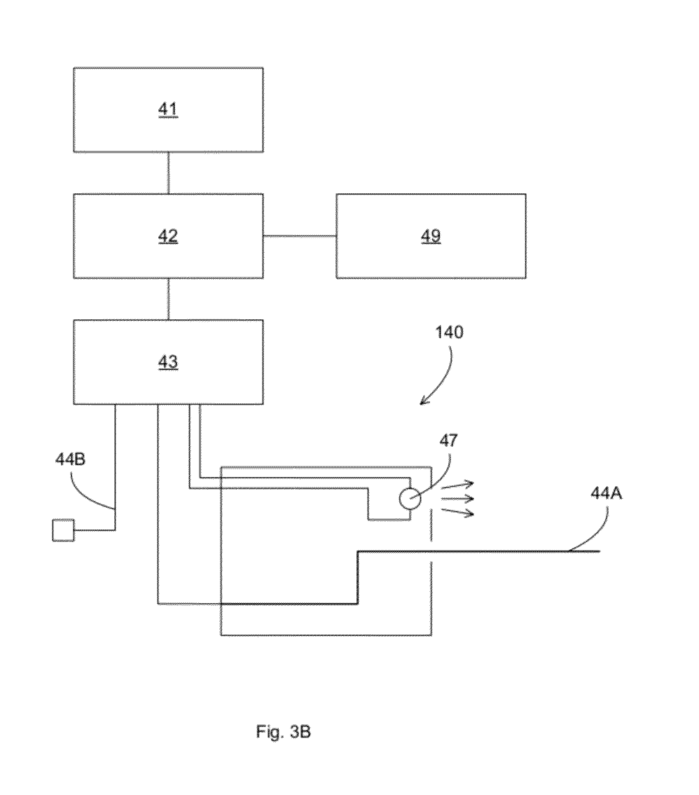

FIGS. 3A-3B illustrate exemplary stimulation systems according to some embodiments of the present invention. In FIG. 3A, an electrode 40 comprises an electrical conduit 44A and a laser light source 45 to deliver a combination of an electrical current stimulation and a laser beam stimulation to a skin point on a patient. The electrical conduit 44A and the laser light source 45 (such as a laser diode) are connected to a power source 43 to provide a desired current to the electrical conduit 44A and to power the laser diode 45. The power source 43 also provides a neutral electrical conduit 44B to complete the circuit from the electrical conduit 44A. The laser diode 45 is designed to deliver a laser beam to the same skin point directed to be the electrical conduit 44A, and can comprise a focused laser beam to the same skin point. An optional light source 47 can be included to illuminate the skin point.

A controller 42 is coupled to the power source 43 to control the parameters of the electrical current and the laser light source, such as the intensity, pulse frequency, and pulse waveform. The controller 42 can offer automatic controls, allowing the therapist to focus on the stimulation treatment. The controller 42 can also offer manual controls, allowing the therapist to set the parameters according to the patient's level of discomfort. The controller 42 can be coupled to a display monitor 41, which provides visual indications for the skin points to be stimulated. An optional acupuncture point finder or a low resistance measurement system 49 is coupled to the controller 42 (or to the monitor 41) to identify the skin points to be stimulated. Alternatively, the system 49 can comprise stored locations of to-be-stimulated skin points based on anatomy landmark and based on successful trials in many individuals. A computer program can be included in the controller 42 to provide guidance and direction to a therapist performing the stimulation. For example, the display 43 can display all the skin points to be stimulated, based on a measurement from the low resistance measurement system 49. The controller 42 then can highlight a skin point to be stimulated next on the monitor, prompting the therapist to perform stimulation on that highlighted skin point. The already-stimulated skin points and the to-be-stimulated skin points can also be indicated distinguishably, allowing a visual inspection of the stimulation progress.

In some embodiments, the stimulation system can comprise a current stimulation. In FIG. 3B, an electrode 140 comprises an electrical conduit 44A to deliver an electrical current stimulation to a skin point on a patient. The electrical conduit 44A is connected to a power source 43 to provide a desired current to the electrical conduit 44A.

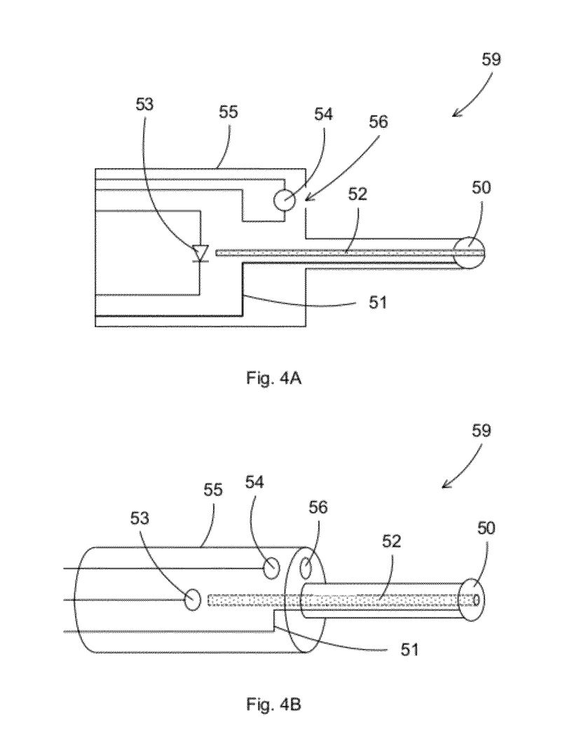

FIGS. 4A-4B illustrate exemplary electrodes according to some embodiments of the present invention. FIG. 4A shows a cross section view and FIG. 4B shows a perspective view of an exemplary electrode 59. The electrode 59 has a ball shaped tip 50 of metal, is hollow in the middle with a straight fiber optic light conductor 52 from a laser diode 53 located in the pen shaped handle 55 to the opening in the center of the ball shaped electrode. Electrical conduit 51 is provided to the metal ball shape electrode. A blue led 54 is located in the handle, and provide illumination through aperture 56. The illustrated electrodes are configured for a delivery of a combination of electrical and light stimulation, but can also be used for electrical stimulation without the laser stimulation. Alternatively, the electrode can comprise only the electrical conduit 51 connected to the ball shaped tip 50, to be used for electrical stimulation without a combination with laser stimulation.

In some embodiments, the present stimulation is used for treating addiction, such as smoking addiction. The present stimulation for smoking cessation is simple and inexpensive, averagely requiring 1 or 2 treatment sessions for 10 to 20 minutes. The present stimulation for smoking cessation is also proven to be effective, achieving long term (2 years) smoking abstinence for a large percentage (about 50%) with an average of just 1 or 2 treatment sessions. In combination with counseling sessions, the present stimulation treatment can be very effective to aid in smoking cessation. The present stimulation treatment can also aid in smoking cessation without rely on medications with potential long term side-effects.

In some embodiments, the present invention discloses a smoking cessation method using consultation meetings and a computer assisted electric or preferably photoelectric stimulation of defined regions in the ears and on the nose. The electric stimulation of ear regions can influence brain regions to modify a person habits and behaviors. The consultation meetings can provide counsel to assist in maintaining the smoking cessation resulted from the stimulation treatment, and to guard against the temptation of returning to the smoking habit.

The present stimulation treatment can be effective as an aid for smoking cessation, showing comparable results to other smoking cessation methods, but with considerable less time and cost effort. In addition, long term efficacy and safety is also tested and proven in larger groups of smokers for over 2 years. The present stimulation treatment can be effective for a wide range of smoker addiction, including males and females; young and old (average 44 years old); and low, moderate and highly addicted (average 30 years of smoking with 25 cigarettes daily).

In some embodiments, a complete treatment comprises a screen to determine the level of addiction, and a computer assisted sequential application of a stimulation of the defined skin areas on the ears and nose with an electric DC-current with or without a red laser light. The stimulation treatment shows some minor discomforts, but within the acceptable tolerance. There can be some mild side effects, which are generally only temporary.

In some embodiments, the present stimulation treatment comprises a parameter determination and a computer controlled and guided sequential application of a stimulation of defined skin areas in both ears and on the nose with an electric DC-current with or without a red laser light source. The treatment points in the ear are developed from ear acupuncture experience but the method also uses areas in the ear that are not described as acupuncture points. These areas were found to have reduced electrical skin resistance in subjects with drawal symptoms and can be identified with an Ohmic resistance measurement. It was suggested that that electric stimulation of ear regions can influence certain areas in the brain, potentially changing the harmful habits.

In some embodiments, the present stimulation treatment employs short treatment durations, in order of 10 minutes on the average per treatment session. In some embodiments, the skin point stimulations utilize a sealed stimulation device, comprising an application electrode which produces a DC signal (for example, 160 DCV, 3 mA maximum current). Preferably, within the head of the application electrode is a pulsating or modulated laser beam (for example, 650 nm, 1.5 mW, 10 Hz). The area surrounding the electrode is optionally illuminated with a blue light to improve visibility for the therapist. The stimulation device is controlled by a controller (for example, a computer running a software program) that controls the intensity, duration and electric curve of the DC current as well as the laser.

The duration and the stating intensity of the stimulation at the skin points is determined based on the biometrics and nicotine addiction levels of the patients, such as age, health status, number of years smoked, average number of cigarettes smoked per day smoked. The stimulation time per skin point can be less than 20 seconds, preferably between 5 and 20 seconds, with an average time around 10 seconds. The starting intensity of the electric stimulation is less than 3 mA, with an average intensity around 2 mA. The intensity can be reduced or stopped, depending on the discomfort level of the patients.

The stimulation is applied by establishing a safe skin contact between the electrode (delivering a pulsed DC current and a laser beam) and the skin point of the patient. The electrode is preferably a 4 mm ball shaped electrode, delivering a DC current impulse and a modulated low power focused laser light impulse through a laser diode in the middle of the electrode. The electrode is applied manually following a computerized map of stimulation areas on both ears and the philtrum of the nose. A neutral electrode is connected to other parts of the patient, for example, through the patient holding the neutral electrode in one hand.

In some embodiments, a computer program provides guidance, showing the skin points to be stimulated from point to point in the standardized sequence. For example, the skin point to be stimulated next is highlighted on the display monitor, guiding the therapist in the stimulation process. The skin points that have been stimulated can also be identified, for example, by displaying with a different color or shade. The nose points are stimulated at the end of the procedure after the ear points. In some embodiments, 39 skin points are selected for stimulation, located on both ears and on the philtrum of the nose. The skin points can be identified by an acupuncture-point finder or a conventional tool to measure reduced Ohmic resistance, for example, on the ears of smokers in withdrawal modes. Preferably, the skin points are identified based on anatomy landmark and based on trials and experiments. The average duration of the skin stimulation process is around 10 minutes.

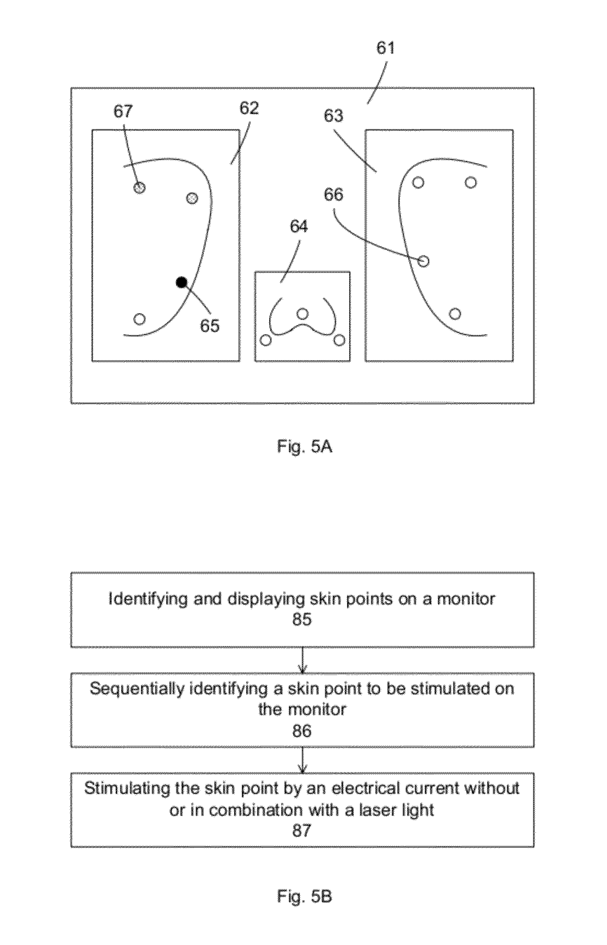

FIG. 5A illustrates an exemplary display according to an embodiment of the present invention. A monitor 61 shows the areas to be stimulated, such as left ear region 62, right ear region 63, and nose region 64, together with the stimulation skin points 65, 66, 67. The skin points preferably can be identified by using the anatomical landmarks shown on the monitor. The skin points can be predetermined, for example, the skin points can be the points that showed in trials to be useful for smoking cessation in many individuals. Alternatively they can be found using an acupuncture point finder or an impedance measurement device, which are then transmitted to a controller to be displayed on the monitor 61. The stimulation skin points can be displayed distinguishably, showing skin points 67 that have been stimulated, skin points 66 that are to be stimulated, and skin point 65 ready to be stimulated next. The display of the distinguished skin points can help automated the stimulation process, keeping track of the stimulation process and progress, and allowing the therapist to focus on performing the stimulation.

FIG. 5B illustrate an exemplary stimulation processes according to an embodiment of the present invention. In operation 85, the skin points are identified and displayed on a monitor. In operation 86, the skin points are sequentially specified from point to point, as an aid to a therapist to know which skin point to be stimulated next. In operation 87, the skin point specified on the monitor is stimulated by an electrical current, or preferably an electrical current in combination with a laser light.

In some embodiments, the present stimulation treatment comprises a first screening session. For example, the screening session can determine medical and smoking history, biometric data and level of nicotine dependence of the patient. Based on the screening data, a number of treatment sessions together with parameters for the stimulation, can be determined.

For example, the screening session includes a measure of the level of nicotine dependence (preferably defined by the FTND score), which leads to the number of treatment sessions for an effective smoking cessation treatment. In general, for low to moderate nicotine addiction, 1-3 treatment sessions are recommended, and for high nicotine addiction, 5 treatment sessions are recommended. In some embodiments, the second treatment can be performed in about 2 weeks or less after the first treatment. Additional treatments can be scheduled within 6 months after the first treatment. Optional counseling sessions are scheduled to assist the patients in reduction of the smoking dependency.

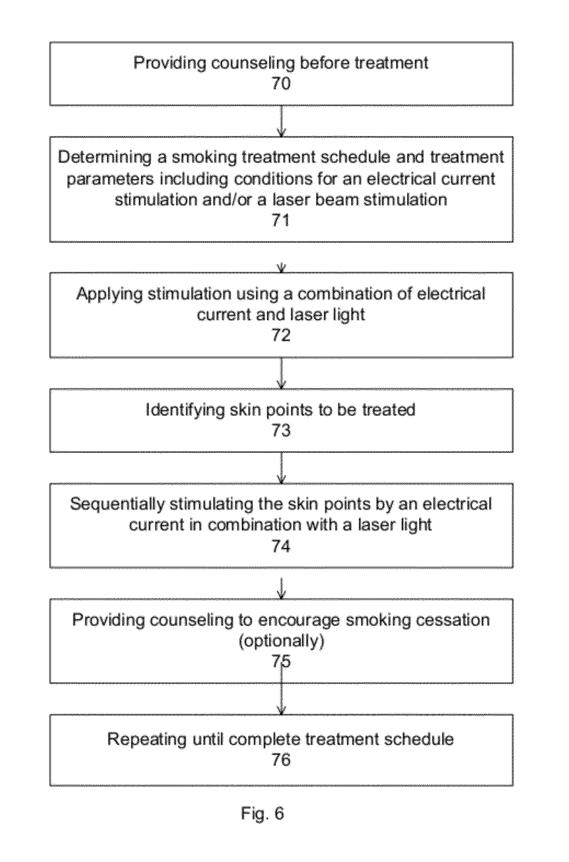

FIG. 6 illustrates exemplary overview of a treatment process according to an embodiment of the present invention. At a first meeting, a short counseling session is conducted after showing a standard video illustrating the treatment process (operation 70). Then the patient's health and level of nicotine addiction are assessed to determine a treating schedule and treating parameters (operation 71). For example, a high level of nicotine addiction, exemplified by the number of cigarettes smoked per day or the time that the patient has been smoking, can be used to determine the number of treatment sessions. In general, a low to medium level of nicotine addiction requires 1 to 2 treatment sessions, while a high level of nicotine addiction requires 4 to 5 treatment sessions. Further, the patient's addiction level can be used to set the stimulation time per stimulation point, with higher addition level requiring a longer stimulating time. The stimulation time is typically less than 20 seconds per point, and is about 10 seconds on the average. The patient's health can be used to determine an initial intensity for the electric current stimulation, which can be adjusted during the stimulation session based on the patient's discomfort level. The current intensity is typically less than 20 mA, and is about 2-3 mA on the average. In some embodiments, the assessment can also be used for determining the stimulation methodology, such as a stimulation using electrical current, or a stimulation using a combination of electrical current and laser light.

In operation 72, the stimulation using electrical current is performed or a combination of electrical current and laser beam is performed using a combination of an electrical current and a laser beam. In operation 73, the skin points are first determined, based on acupuncture points or low resistance skin points. In operation 74, an electrode is then sequentially applied to the skin points, with the stimulation parameters determined from the health and addiction assessment. The stimulation is repeated until all the skin points are stimulated. A computer program can help in guiding from skin point to skin point in the stimulation process.

In operation 75, counseling is optionally provided to encourage smoking cessation. Addiction can be driven by the environment, such as stress or social engagement, and thus counseling can help re-enforcing the smoking abstinence obtained from the stimulation. In operation 76, the patient can return for subsequent treatment sessions to complete the treatment schedule, with the number of treatment sessions determined from the patient's addiction level.

### Example

A large number of participants (150+) were treated for smoking cessation using the present stimulation treatment. The participants are then monitored at 3 months, 6 months, and 2 years after treatment.

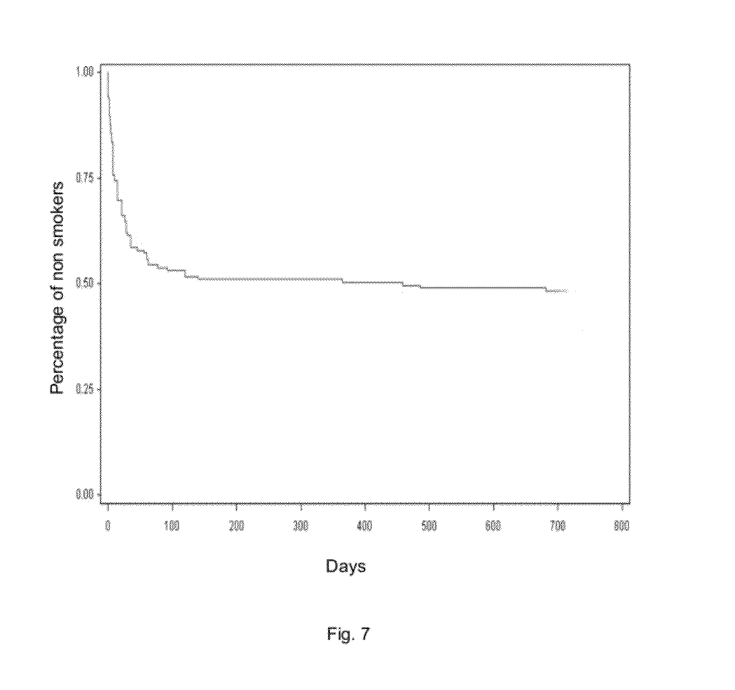

FIG. 7 illustrates an exemplary result according to an embodiment of the present invention. After the first treatment, 89.7% of the participants stopped smoking for more than two days, and 76.7% stopped for more than a week. After one year 49.3% were abstinent, after 2 years 47.9% remained abstinent. Given the low cost and simplicity of the present treatment, an abstinence rate of almost 50% for 2 years can be considered highly successful.

Further, the success rate could be much higher, since a majority of the participants do not adhere to the full number of recommended treatments. For example, on average, the participants completed only about a third of the recommended treatments. Grouping separately, long term successful abstinent participants were 3 times more compliant in completing recommended treatments than those that relapsed.

In addition, there is no adverse effect on the relapsed smokers. Relapsed smokers decreased their overall cigarette consumption initially, but with time, their consumption rate approached their pre-treatment levels. No relapsed smokers consume more cigarettes than before the treatment.

The present invention may be embodied in other specific forms without departing from the spirit or essential attributes thereof, and it is therefore desired that the present embodiments be considered in all respects as illustrative and not restrictive, reference being made to the appended claims rather than to the foregoing description to indicate the scope of the invention.

What is claimed is:

1. A method to assist in smoking cessation, comprising
exhibiting skin points that showed in trials to be useful for smoking cessation in many individuals; and
stimulating the skin points by an electrical current in combination with a laser light.

2. A method of claim 1, wherein the skin points comprise skin points located in ear area.

3. A method of claim 1, wherein the skin points are located in ear and nose areas.

4. A method of claim 3, wherein skin points on ear area are selected for stimulation before skin point on nose area.

5. A method of claim 1, wherein the electrical current comprises a pulsed DC current having frequency between 0.5 and 2 Hz with a voltage less than 160V.

6. A method of claim 1, wherein the electrical current comprises a pulsed DC current having a peak pulse between 1 and 3 mA for less than 0.2 second, followed by a constant current between 0.5 and 3 mA for less than 1 second, and followed by a zero current for less than 1 second.

7. A method of claim 1, wherein the laser light comprises a pulsed red laser light having frequency less than 10 Hz and having power less than 5 mW.

8. A method of claim 1, wherein the skin points are identified by anatomy landmark.

9. A method of claim 1, further comprising
determining a degree of nicotine dependence;
setting a stimulation time for the skin points based on the degree of nicotine dependence.

10. A method of claim 1, further comprising
displaying multiple skin points on a display monitor;
identifying a next skin point to be stimulated.

11. A method of claim 1, further comprising
determining a degree of nicotine dependence;
prescribing a number of treatment sessions based on the degree of nicotine dependence.

12. A method of claim 1, further comprising
applying an illumination in a vicinity of the skin point.

13. A method to assist in smoking cessation, comprising
identifying multiple skin points on ear and nose locations that showed in trials to be useful for smoking cessation in many individuals; and
sequentially stimulating the multiple skin points by an electrical current in combination with a laser light.

14. A method of claim 13, further comprising
displaying the multiple skin points on a display monitor;
automatically identifying a next skin point to be stimulated.

15. A method of claim 13, further comprising
determining a degree of nicotine dependence;
automatically prescribing a number of treatment sessions and a stimulation length for the skin points based on the degree of nicotine dependence.

16. A method of claim 15,
wherein the electrical current comprises a pulsed DC current having frequency between 0.5 and 2 Hz with a voltage less than 160V;
wherein the electrical current comprises a pulsed DC current having a peak pulse between 1 and 3 mA for less than 0.2 second, followed by a constant current between 0.5 and 3 mA for less than 1 second, and followed by a zero current for less than 1 second;
wherein the laser light comprises a pulsed red laser light having frequency less than 10 Hz and having power less than 5 mW;
wherein the electrical current and laser light are automatically determined based on the degree of nicotine dependence.

17. A method of claim 13, further comprising providing counseling to reduce smoking relapse.

18. A method of claim 13, wherein 2 year smoking abstinence is achieved after 1 or 2 treatment sessions.

19. A method to assist in smoking cessation, comprising
exhibiting multiple skin points on ear and nose locations that showed in trials to be useful for smoking cessation in many individuals, wherein the multiple skin points are displayed on a monitor;
automatically identifying, by a controller, a skin point to be stimulated on the monitor;
stimulating, by an operator, the identified skin point by an electrical current;
repeating the sequence of identifying and stimulating for the multiple skin points.

20. A method of claim 19, wherein the electrical current comprises a pulsed DC current having frequency between 0.5 and 2 Hz with a voltage less than 160V, and having a peak pulse between 1 and 3 mA for less than 0.2 second, followed by a constant current between 0.5 and 3 mA for less than 1 second, and followed by a zero current for less than 1 second.
